(19) United States
(12) Patent Application Publication
(10) Pub. No.: US 2012/0164187 A1
(43) Pub. Date: Jun. 28, 2012

(54) BIOACTIVE GLASS FOR USE IN CONDITIONS RELATING TO BONE INFECTIONS

(76) Inventors: Fredrik Ollila, Turku (FI); Jimmy Lucchesi, Turku (FI); Nina Lindfors, Grankulla (FI); Pekka Hyvonen, Oulu (FI); Hanna Liuke, Turku (FI)

(21) Appl. No.: 13/380,465

(22) PCT Filed: Jun. 29, 2010
(86) PCT No.: PCT/FI2010/050558

§ 371 Date: Mar. 15, 2012

(30) Foreign Application Priority Data
Jun. 29, 2009 (EP) 09008475.7
Jun. 29, 2009 (FI) 20090257
Nov. 16, 2009 (FI) 20096181

(51) Int. Cl. A61K 33/42; A61K 9/14; A61P 19/00; A61K 35/12

(52) U.S. Cl. 424/400; 424/601; 424/93.7; 424/602

(57) ABSTRACT

The present invention relates to a bioactive glass having the composition of SiO₂ 44-65 wt-% of the final total weight, Na₂O 5-26 wt-% of the final total weight, CaO 10-25 wt-% of the final total weight, K₂O 0-1 5 wt-% of the final total weight, MgO 0-6 wt-% of the final total weight, B₂O₃ 0-4 wt-% of the final total weight, and P₂O₅ 0-7 wt-% of the final total weight, for use in the long- and short-term prevention and/or treatment of conditions relating to or caused by bone infections, provided that the total amount of Na₂O and K₂O is 10-30 wt-% of the final total weight, and that any source of oxygen capable of releasing oxygen in the form of molecular oxygen or reactive oxygen species, is absent.

### FIELD OF THE INVENTION

The present invention relates to a bioactive glass useful in the long- and short-term prevention and/or treatment of bone infections and bone infection-related inflammatory reactions, and in promotion of tissue healing and/or regeneration relating or caused by bone infection. The invention relates also to uses of the said bioactive glass.

### BACKGROUND OF THE INVENTION

Osteomyelitis is a disease which is heterogeneous in its pathophysiology, clinical presentation, and management. It is felt to be one of the most difficult-to-treat infectious diseases. Progressive bony destruction and the formation of sequestra are hallmarks of osteomyelitis. The disease may be acute, subacute, or chronic. Chronic osteomyelitis may appear as such at the initial presentation; not all patients show progression through the three phases.

Chronic osteomyelitis is a severe, persistent, and sometimes incapacitating infection of bone and bone marrow. It is often a recurring condition because it is difficult to treat definitively. This disease may result from (1) inadequate treatment of acute osteomyelitis; (2) a hematogenous type of osteomyelitis; (3) trauma, (4) iatrogenic causes such as joint replacements and the internal fixation of fractures; (5) compound fractures; (6) infection with organisms, such as Mycobacterium tuberculosis and Treponema species; and (7) contiguous spread from soft tissues, as may occur with diabetic ulcers or ulcers associated with peripheral vascular disease.

The ends of long bones are the most common locus of infection, and Staphylococcus aureus is the most common infective organism involved. In the presence of antibiotic-resistant bacteria, such as MRSA, the treatment by antibiotics is more demanding, and the relapse may be up to 45% in those cases. Treating chronic cases may easily become problematic which may lead to a very long and complex treatment chain consisting of multiple treatments by oral and intravenous antibiotics, surgery, and hyperbaric oxygen (HBO). The end result after extensive care may nevertheless be the amputation of part of the limb or death in the worst case. Relapse of osteomyelitis may occur many years after the initial treatment.

The treatment of chronic osteomyelitis includes debridement of the dead infected tissue, obliteration of dead space, osseous repair, adequate soft tissue coverage, and systemic antibiotics. Intravenous antibiotics are used commonly in the treatment of chronic osteomyelitis. Part of the pathological process in this chronic condition is the formation of avascular, necrotic areas of bone (sequestra) which harbour bacteria. As these lack a blood supply, antibiotics cannot reach them and surgical intervention is required. In general, chronic osteomyelitis is considered as a ‘surgical illness’ and debridement plays the most important part of the treatment.

After saucerisation of osteomyeltic bone, muscle flap can be transpositioned to obliterate the bony defect, and antibiotic-impregnated polymethyl methacrylate (PMMA) beads are used to sterilize and temporarily maintain dead space. The cement beads are usually removed within 2-4 weeks and replaced with a cancellous bone graft in certain cases. Other bone graft substitute materials have also been used to obliterate the cavity; however, the problem of many conventional bone graft substitutes is that they are prone to infections because they allow microbial growth on their surface at the implantation site.

There are several solutions available to treat osteomyelitis but they are based on local delivery of antibiotics to the implantation site, e.g. the bioabsorbable drug delivery systems described by Lin et al., “Evaluation of a biodegradable drug delivery system for chronic osteomyelitis,” 38th Annual Meeting, ORS, Washington D.C., Feb. 17-20, 1992; Robinson et al. “Preparation and degradation of a biodegradable gentamycin delivery system for the treatment of osteomyelitis”, 38th Annual Meeting, ORS, Washington D.C., Feb. 17-20, 1992; Garvin, et al., “Treatment of Canine Osteomyelitis with a Biodegradable Antibiotic Implant.” 38th Annual Meeting, ORS, Washington D.C., Feb. 17-20, 1992; and Wei et al., “A bioabsorbable delivery system for antibiotic treatment of osteomyelitis,” J. Bone Joint Surg. 73B:246-252, 1991.

Di Silvio and Bonfield describe a drug delivery system comprising gelatin for the combined release of therapeutic levels of both gentamycin and growth hormone in “Biodegradable drug delivery system for the treatment of bone infection and repair”, Int. Conf. Adv. Biomater. and Tissue Eng., June 14-19, Capri, Italy, Book of Abstracts, p. 89-90, 1998. This system releases gentamycin only up to 14 days, which is in many cases too short of a time because effective healing of an osteomyelitis may need antibiotic treatment for at least several weeks (see e.g. L. Dahl et al, Scand. J. Infect. Dis., 30:573-577, 1998). Additionally gelatine based systems are mechanically weak and cannot be used in the form of bone fracture fixation implants. Also, animal-based biomaterials, including gelatin, have aroused concern of the risk of delivering animal-based diseases, such as viral infections, into human patients. In addition, the release of bone growth promoting factor (growth hormone) was limited to 2 weeks as well, which is far too short a time for proper new bone formation, which in the case of cancellous bone is at least 6 weeks.

WO 2008/033221 describes a biodegradable bone replacement material for the treatment of bone defects and delivery of antibiotic compounds, particularly for treating bone infections, comprising calcium sulphate hemihydrate, calcium sulphate dihydrate, an antibiotic mixture comprising a tetracycline compound and an ansamycin compound. Methods for treating, repairing or augmenting an osseous defect using the bone replacement material are also provided.

Bioactive glass (BAG) is a known bioactive material. Unlike with most other bioactive materials, it is easy to control the manufacturing properties of bioactive glass, the rate of its chemical reactions, and the biological response caused by it simply by altering the chemical composition of bioactive glass itself. Bioactive glass has been used in different types of implants, such as bone fillers/substitutes, bone growth promoting materials, middle ear prostheses etc. Specifically, BAG S53P4, a bioactive glass available from Vivoxid Ltd, Finland, has proven to promote bone formation many years after implantation while slowly resorbing away (Peltola et al. “Bioactive glass S53P4 in frontal sinus obliteration: a long-term clinical experience” Head & Neck 28:834-841, 2006). The healing process progresses from a fibrous tissue phase to bone formation with scattered fibrous tissue and bony obliteration maintaining BAG granule remnants.

Bioactive glass powders have been shown in vitro to present bacterial growth-inhibiting properties on 17 clinically important anaerobic bacterial species (Lepparanta et al. “Antibacterial effect of bioactive glasses on clinically important anaerobic bacteria in vitro” J Mater Sci: Mater. Med., 19:547-51, 2008). In another in vitro study, glass powders of several composition presented similar growth-inhibition properties towards 29 clinically important aerobic bacterial species (Munukka et al. “Bactericidal effects of bioactive glasses on clinically important aerobic bacteria” Mater Sci: Mater. Med., 19:27-32, 2007). BAGs usually release ions such as sodium, calcium, phosphate, and silicate in aqueous conditions and their release elevates the pH and osmotic pressure of the environment. The optimal pH of all the bacteria tested is close to neutral. Thus, the increase in pH could partly explain the growth inhibition. Another factor may be the high concentrations of calcium and alkalis likely to be released from the BAG that could cause perturbations of the membrane potential of bacteria. The release of ions from the bioactive glass is preferably slow enough for not to irritate the cells and tissues in contact with the implant or to interfere with the normal inflammatory cell response, especially macrophages, at the implantation site.

Leaching of alkali, and alkaline earth ions leads to a fast increase in pH around the glass, which has been shown to depend on the composition of the glass. Glass S53P4 has in a simulated body fluid shown an increased pHₘₐₓ value of 11.65. The high pH, and the subsequent osmotic effect cased by dissolution of the glass has been suggested to partly explain the antibacterial properties observed for BAGs. Comparing bactericidal effects of different BAGs, glass S53P4 has been shown to be the most effective, with the fastest killing or growth inhibitory effect. This antibacterial effect has been observed in vitro for all pathogens tested, including the most important aerobic and anaerobic pathogens, as well as very resistant bacteria.

Bone infection usually leads to an inflammatory response manifested in the recruiting of inflammatory host cells such as neutrophils, monocytes and macrophages, but, surprisingly, also osteoblasts. It has been demonstrated that bacterial challenge of osteoblasts during bone diseases such as osteomyelitis induces cells to produce a key inflammatory chemokine that can direct appropriate host responses or may contribute to progressive inflammatory damage (Marriott et al., “Osteoblasts produce monocyte chemoattractant protein-1 in a murine model of Staphylococcus aureus osteomyelitis and infected human bone tissue” Bone, 37:504-512, 2005). The inflammatory response, and the bone vasculature damage can lower the local pH considerably, and the healing process may be further prolonged. Bioactive glass can increase the pH to a physiologically more favorable level, where healing can better occur and the tissue can recover more rapidly once the infection is cured.

Vascularisation plays an important role in the healing of any tissue, including bone tissue. Osteomyelitis can be the cause of bone necrosis, but on the other hand, bone that becomes necrotic for any other reason (i.e. trauma, illness or radiation therapy) is highly susceptible to infection. Therefore, securing a constant blood supply to the damaged tissue is vital to ensure a proper immunologic response when needed, and a rapid angiogenesis phenomenon would thus be required. Angiogenesis is also an essential part of the healing process by supplying oxygen and nutrients for the growing tissue. The earlier sufficient vascularisation occurs, the faster new tissue formation is seen.

Bioactive glass has been reported to contain angiogenic properties in vitro by R. Day “Bioactive glass stimulates the secretion of angiogenic growth factors and angiogenesis in vitro” Tissue Engineering 11:768-777, 2005 and Leu and Leach, “Proangiogenic potential of a collagen/bioactive glass substrate” Pharmaceutical Research 25:1222-1228, 2008, and in an animal study by Leu et al. “Angiogenic response to bioactive glass promotes bone healing in an irradiated calvarial defect” Tissue Engineering: Part A 14:1-9, 2008. In all three studies the angiogenic response was observed by using low concentrations of bioactive glass. Furthermore, document US 2006/233887 describes the use of low concentrations (0.00001 to 10 wt-%) of bioactive glass in stimulating vascularisation.

Several different bioactive glass compositions have been presented in the art, also for use in bones. WO 2008/000888 describes an implant comprising a source of oxygen capable of releasing oxygen in the form of molecular oxygen or reactive oxygen species, and a material selected from the group consisting of biodegradable and/or bioactive glass, sol-gel produced silica and mixtures thereof. This implant combines peroxides with ceramics to treat infected tissues. Document US 2004/0009598 presents the use of bioactive glass compositions to stimulate osteoblast production, but does not mention any effect on infections. Document WO 99/16423 discloses the use of biologically active glass as a drug delivery system.

P. Stoor et al. “Antibacterial effects of a bioactive glass paste on oral micro-organisms”, Acta Odontol. Scand. 56:161-165, 1998, and in references cited therein describes a bioactive glass to have an antibacterial effect on oral micro-organisms.

U.S. Pat. No. 6,579,533 describes synthetic drug delivery materials and implants comprising (a) a synthetic bioabsorbable polymeric matrix; (b) an antibiotic phase dispersed into said polymeric matrix; and (c) antibacterial, bioabsorbable, bioactive glass, dispersed into said polymeric matrix for the promotion of bone growth.

Despite intensive treatment with both surgery and prolonged parenteral administration of antimicrobial agents, the course of the illness may result in persistence or relapse of the infection and suggests that better treatment options are desperately needed. A bone graft material that can prevent osteomyelitis from recurring and simultaneously offer an osteoconductive, bioresorbable scaffold to support new bone and capillary formation would be considered as a clear improvement to current treatment pathways.

### OBJECT AND SUMMARY OF THE INVENTION

An object of the invention is to minimise or even eliminate the problems existing in the prior art.

One object of the present invention is to provide a material useful in preventing and managing microbial infections in bones.

Another object of the present invention is to provide a material useful in treating and replacing diseased bone tissue in mammals.

Another object of the present invention is to provide a material useful in enhancing the healing or regeneration of bone tissue as well as surrounding tissue compromised due to delayed access to oxygen physiologically provided by the forming capillary network.

In order to achieve the above-mentioned objects the present invention is characterised in what is defined in the characterising parts of the independent claims presented hereafter.

The present invention relates to a bioactive glass having the composition of
SiO₂ 44-65 wt-% of the final total weight,
Na₂O 5-26 wt-% of the final total weight,
CaO 10-25 wt-% of the final total weight,
K₂O 0-15 wt-% of the final total weight,
MgO 0-6 wt-% of the final total weight,
B₂O₃ 0-4 wt-% of the final total weight, and
P₂O₅ 0-7 wt-% of the final total weight,
   for use in the long- and short-term prevention and/or treatment of conditions relating to or caused by bone infections,
   provided that the total amount of Na₂O and K₂O is 10-30 wt-% of the final total weight, and that any source of oxygen capable of releasing oxygen in the form of molecular oxygen or reactive oxygen species, is absent.

### DEFINITIONS

The terms used in this application, if not otherwise defined, are those agreed on at the consensus conference on biomaterials in 1987 and 1992, see Williams, DF (ed.): Definitions in biomaterials: Proceedings of a consensus conference of the European Society for Biomaterials, Chester, England. Mar. 3-5, 1986. Elsevier, Amsterdam 1987, and Williams D F, Black J, Doherty P J. Second consensus conference on definitions in biomaterials. In: Doherty P J, Williams R L, Williams D F, Lee A J (eds). Biomaterial-Tissue Interfaces. Amsterdam: Elsevier, 1992.

In this application, by bioactive material is meant a material that has been designed to elicit or modulate biological activity. The term biodegradable in this context means that it is degradable upon prolonged implantation when inserted into mammalian body. By biomaterial is meant a material intended to interface with biological systems to evaluate, treat, augment or replace any issue, organ or function of the body. By biocompatibility is meant the ability of a material used in a medical device to perform safely and adequately by causing an appropriate host response in a specific location. By resorption is meant reduction/disintegration of biomaterial because of cellular activity or simple dissolution.

Implants in this context are meant to comprise any kind of implant used within the body, such as artificial organs and parts thereof, joint implants, internal fixation devices, devices used for reconstruction or replacement of bones and tissues, devices used for supporting and/or stimulation of tissue healing or regeneration, devices used for filling defects in bones and materials used as sealant or posts in the root canal of a tooth. Depending on the application and purpose of the implant materials, they are expected and designed to be biocompatible and exhibit either longevity or controlled degradability in the body. The optimal degradation rate is directly proportional to the renewal rate of the tissue. In the case of bone tissue, a considerable proportion of the implant is preferably degraded by 6 weeks in the tissue. In cases where physical support to the healing tissues is desirable the degradation rate might be several months or even one year. In some embodiments of the invention the degradation rate may even be nonexistent.

Infection in this context comprises various infections in bone or bone cavities within the human body. Infections may occur inside the body, subcutaneously or on the surface of the body.

Wt-% stands for weight percentage in this description, typically calculated from the total weight of the composition.

### DETAILED DESCRIPTION OF THE INVENTION

The present invention relates to a bioactive glass having the composition of
SiO₂ 44-65 wt-% of the final total weight,
Na₂O 5-26 wt-% of the final total weight,
CaO 10-25 wt-% of the final total weight,
K₂O 0-15 wt-% of the final total weight,
MgO 0-6 wt-% of the final total weight,
B₂O₃ 0-4 wt-% of the final total weight, and
P₂O₅ 0-7 wt-% of the final total weight,
   provided that the total amount of Na₂O and K₂O is 10-30 wt-% of the final total weight, and that any source of oxygen capable of releasing oxygen in the form of molecular oxygen or reactive oxygen species, is absent,
   for use in the long- and short-term prevention and/or treatment of conditions relating to or caused by bone infections.

The material according to the present invention is thus a bioactive glass composition that does not comprise any source of oxygen (as disclosed in WO 2008/000888) and that can be used as such, without any other agents or microencapsulation to treat infected bones and surrounding tissues, where the damage has been caused by the infected bone. The invention thus concerns infected bones and problems caused by them.

A source of oxygen capable of releasing oxygen in the form of molecular oxygen or reactive oxygen species that is disclaimed from the present invention relates to materials that are capable of releasing for example gaseous oxygen (O2) or ozone (O3), or hydroxyl ions, hydroxyl radicals or oxygen radicals. Such material can naturally also release oxygen in a mixture of these forms and can be for example urea peroxide, calcium peroxide, magnesium peroxide, sodium percarbonate or potassium monopersufate. It is clear to a person skilled in the art that the oxides constituting the bioactive glass do not qualify as sources of oxygen in the present sense.

The current invention solves several problems in the prior art. The bioactive glass according to the present invention inhibits the infection in the bone and surrounding tissue, while at the same time improves the growth and regeneration of the tissues in which it is situated. Ions released from the bioactive glass produces marked increase in the amount of ions dissolved in tissue fluid, which is believed to promote cell and tissue growth at the bioactive glass surface and its immediate vicinity, thus adding to the known osteoconductive and osteopromotive effect of bioactive material. Also, the ions released from the material enhance and stimulate the capillary formation and give rise to an antibacterial effect.

According to an embodiment of the invention, the bioactive glass has the composition of
SiO₂ 53 wt-%,
Na₂O 23 wt-%,
CaO 20 wt-% and
P₂O₅ 4 wt-%.

This glass is known as S53P4 glass.

Surprisingly, this bioactive glass S53P4 alone, without antibiotics or any other additionally added active components that aid tissue healing, can heal bone that has been affected by chronic osteomyelitis. The clinical effect can be assumed to be through the combined effect of having antibacterial, bioactive and osteopromotive properties in the bioactive glass which thus prevents relapse of the infection and at the same time generates rapid bone formation allowing for accelerated healing. This clinical effect has been recently seen where S53P4 was used to fill bone voids of patients who had suffered from chronic osteomyelitis (see the Experimental part below). Based on the knowledge of a skilled person in this field, it is believed that the same effect can be achieved with other glasses having their composition within the above-mentioned range. It is therefore an aspect of the present invention that the composition can be used without the presence of any other therapeutically active agent. More specifically, the composition can be used in the absence of antibiotics, to manage an infection. On the other hand, any other therapeutically active components than antibiotics may be included into the composition to further enhance the overall beneficial tissue response of the bioactive glass. These components include stem cells, growth factors (such as bone morphogenic proteins, BMPs), etc.

According to an embodiment of the invention, the glass is thus used in combination with at least one therapeutically active component, provided that the therapeutically active component is not an antibiotic.

Bioactive glass S53P4 resorbs slowly and is replaced by new bone in a process that takes many years. With current treatment options, osteomyelitis may relapse several years after the initial treatment. The slow constant resorption of S53P4 glass ensures that active bone formation takes place at the defect site, and that no dead space will be formed during the process, thus leaving no room for potential defect complications.

The bioactive glass of the present invention is for use in the long- and short-time prevention and treatment of conditions selected from the group consisting of bone infections, bone infection relating tissue healing and bone infection relating tissue regeneration.

According to another embodiment, the bioactive glass is in the form of particles, granules, fibres, tubes, coatings, spheres, or powder. The particles may have a diameter in the range of 0.04-4.0 mm. Preferably, the bioactive glass is in the form of granules. The preferred diameter of the bioactive glass entities is 0.5-2.0 mm. The bioactive glass may also be in the form of an implant. Furthermore, the implant may comprise other constituents, for example the bioactive glass may be embedded in a neutral bioresorbable matrix material suitable for the intended use such as a mixture of polyethylene glycol and glycerol. By neutral, it is meant material that does not take any role in the healing process. The bioactive glass may also be used as a coating material for biodegradable and non-biodegradable implants used in the body that are made of materials such as metals, ceramics, polymers, etc to prevent bacterial films from forming onto the implant surface.

As an additional component of the implant, it is also possible to use pure calcium phosphate CaP, tricalcium phosphate, or calcium sulphate. Hydroxyl apatite, hydroxyapatite, hydroxycarbonated apatite are other possible materials. Moreover, other bioactive ceramic materials or bioactive or biodegradable polymers may be used. Any mixtures of these components can also be used.

According to one embodiment of the invention, the bioactive glass according to the present invention is intended for use in the long- and short-term treatment and/or prevention of chronic infections, such as infections associated with avascularisation of bone, bone necrosis and osteomyelitis. The infection can be acute or chronic. In the case of osteomyelitis for example, the treatment is problematic since the lesion is characteristically ischemic and after treatment/resection there are no more blood vessels to bring oxygen to the lesion site.

Bioactive glass has been shown to promote angiogenesis both in in vitro and in vivo models in low concentrations (see above). The S53P4 bioactive glass, on the other hand, has been observed to give the same effect with high concentrations when used alone in experimental models. The vascularisation and new bone formation was shown to be faster with bioactive glass than with hydroxyapatite, and the initial fibrous tissue formation, which is related to a considerable amount of blood vessels, was reportedly more rapid in bioactive glass filled defects (Peltola et al. “In vivo model for frontal sinus and calvarial bone defect obliteration with bioactive glass S53P4 and hydroxyapatite” J. Biomed. Mat. Res. 58:261-269, 2001).

The bioactive glass according to the present invention can thus be used to sustain the remaining cells until neovascularisation is completed (re-growth of blood vessels). At the same time, the ions released from the bioactive glass are acting as an antimicrobial agent at the surface of the glass, killing infectious cells. The present bioactive glass thus solves the problem encountered with the prior art implant materials. Moreover, no other bone filling material is needed, thus avoiding any risks of contamination by harvested bone.

According to an embodiment of the invention, the bioactive glass is for use in ear, nose and throat, cranio-maxillofacial, orthopaedic or spine surgery. Indeed, the bioactive glass may be used in a wide variety of orthopaedic, neurosurgical, and cranio-maxillofacial surgical operations to prevent osteomyelitis in susceptible patients for example, those patients with prior history of acute or chronic osteomyelitis, and those undergoing an infection other than osteomyelitis, such as a bacteremia.

The invention also relates to a composition comprising bioactive glass having the composition mentioned above and in the independent claims for use in the long- and short-term prevention and/or treatment of conditions relating to or caused by bone infections, provided that the composition does not comprise antibiotics. The invention also relates to a composition comprising bioactive glass having the above-mentioned composition and a therapeutically active agent different from antibiotics.

Furthermore, the invention relates to the use of a bioactive glass having the composition of
SiO₂ 44-65 wt-% of the final total weight,
Na₂O 5-26 wt-% of the final total weight,
CaO 10-25 wt-% of the final total weight,
K₂O 0-15 wt-% of the final total weight,
MgO 0-6 wt-% of the final total weight,
B₂O₃ 0-4 wt-% of the final total weight, and
P₂O₅ 0-7 wt-% of the final total weight,
   provided that the total amount of Na₂O and K₂O is 10-30 wt-% of the final total weight and that any source of oxygen capable of releasing oxygen in the form of molecular oxygen or reactive oxygen species, is absent,
   in the manufacture of an implant for the long- and short-term treatment of a condition relating to or caused by bone infections.

The invention also relates to a method of long- and short-term treatment of a defect relating to or caused by bone infections, comprising the step of inserting bioactive glass having the composition of
SiO₂ 44-65 wt-% of the final total weight,
Na₂O 5-26 wt-% of the final total weight,
CaO 10-25 wt-% of the final total weight,
K₂O 0-15 wt-% of the final total weight,
MgO 0-6 wt-% of the final total weight,
B₂O₃ 0-4 wt-% of the final total weight, and
P₂O₅ 0-7 wt-% of the final total weight,
   provided that the total amount of Na₂O and K₂O is 10-30 wt-% of the final total weight and that any source of oxygen capable of releasing oxygen in the form of molecular oxygen or reactive oxygen species, is absent, in said defect.

In this specification, except where the context requires otherwise, the words “comprise”, “comprises” and “comprising” means “include”, “includes” and “including”, respectively. That is, when the invention is described or defined as comprising specified features, various embodiments of the same invention may also include additional features.

### EXPERIMENTAL PART

The present inventors have successfully treated osteomyelitis in four patients using bioactive glass S53P4 as a bone graft substitute.

### Example 1

The lateral cuneiform (also known as third cuneiform/external cuneiform) intermediate in size between the other two cuneiform bones was severely damaged by osteomyelitis in a patient. The osteomyelitic tissue was thoroughly cleaned through debridement of the dead infected tissue and the dead space was obliterated with bioactive glass S53P4. The patient was fully cured after a 2 year follow-up. The operation was performed at the Helsinki University Hospital.

### Example 2

A patient suffering from chronic two level spondylitis—which is a chronic osteomyelitis of the vertebra—was operated on at the Helsinki University Hospital and after thorough cleaning of the two vertebral bodies that were infected the dead space was obliterated with 16 cc bioactive glass S53P4 in each vertebral body. The patient was healing well after the 6 month follow-up.

### Example 3

A patient with chronic osteomyelitis in the calcaneus or heel bone was operated on and the osteomyelitic tissue was thoroughly cleaned through debridement of the dead infected tissue and the dead space was obliterated with bioactive glass S53P4. After a period of 1.5 years the patient has fully recovered from the chronic osteomyelitis. The operation was performed at the Central hospital of North Carelia (Pohjois-Karjalan keskussairaala).

### Example 4

A diabetes patient with chronic osteomyelitis in one metatarsal bone in the foot was operated on and the osteomyelitic tissue was thoroughly cleaned through debridement of the dead infected tissue and the dead space was obliterated with bioactive glass S53P4. After a period of 1.5 years the patient has fully recovered from the chronic osteomyelitis. The operation was performed at the Central hospital of North Carelia (Pohjois-Karjalan keskussairaala).

### Example 5

Five patients suffering from chronic osteomyelitis were operated from 2007 to 2009 at the Oulu University Hospital (see Table 1). All patients had previously had at least one unsuccessful surgery to treat the infected bone. The follow-up periods varied from 9 to 32 months after bioactive glass obliteration, and no osteomyelitis-related symptoms or complications were observed during the follow-up for any of the patients.

TABLE 1

| | Earlier | Application | |
|---|---|---|---|
| Patient | operations | date | Bacterial culture |
| 1 | 1 | Jan. 9, 2007 | S. aureus |
| 2 | several | Feb. 14, 2008 | Pseudomonas, S. epidermis |
| 3 | several | Jan. 22, 2008 | S. epidermis, Enterobacter |
| | | | cloacae |
| 4 | several | Jun. 18, 2008 | S. aureus, Stenotrophomas |
| 5 | 1 | Feb. 3, 2009 | negative |

1-15. (canceled)

16. A method for prevention and/or treatment of a bone infection, comprising administering to a patient in need of such treatment, a bioactive glass having the following composition:
SiO₂ 44-65 wt-% of the final total weight,
Na₂O 5-26 wt-% of the final total weight,
CaO 10-25 wt-% of the final total weight,
K₂O 0-15 wt-% of the final total weight,
MgO 0-6 wt-% of the final total weight,
B₂O₃ 0-4 wt-% of the final total weight, and
P₂O₅ 0-7 wt-% of the final total weight,
provided that the total amount of Na₂O and K₂O is 10-30 wt-% of the final total weight, and that any source of oxygen capable of releasing oxygen in the form of molecular oxygen or reactive oxygen species, is absent.

17. The method of claim 16, wherein said treatment is for for long- and short-term prevention and treatment of osteomyelitis.

18. The method of claim 16, wherein the glass further comprises a neutral carrier matrix.

19. The method of claim 18, wherein said neutral carrier matrix is a mixture of polyethylene glycol and glycerol.

20. The method of claim 16, wherein said glass is administered in combination with at least one therapeutically active component, provided that said therapeutically active component is not an antibiotic.

21. The method of claim 21, wherein said therapeutically active component is selected from the group consisting of stem cells and growth factors.

22. The method of claim 16, wherein said bone infection is acute or chronic.

23. The method of claim 16, wherein said bioactive glass has the following composition:
SiO₂ 53 wt-%,
Na₂O 23 wt-%,
CaO 20 wt-% and
P₂O₅ 4 wt-%.

24. The method of claim 16, wherein said bioactive glass is in a form selected from the group consisting of particles, granules, fibres, tubes, coatings, spheres and powder.

25. The method of claim 24, wherein the particles have a diameter in the range of 0.04-4.0 mm.

26. The method of claim 16, wherein the bioactive glass is in the form of a coating material on an implant.

27. The method of claim 16, wherein the bioactive glass is in the form of an implant.

28. The method of claim 27, wherein said implant further comprises a component selected from the group consisting of pure calcium phosphate, tricalcium phosphate, calcium sulphate, hydroxyl apatite, hydroxyapatite, hydroxycarbonated apatite, other bioactive ceramic materials, bioactive polymers, biodegradable polymers, hydrogels and mixtures thereof.

29. The method of claim 16, wherein said bioactive glass is administered as part of a surgery selected from the group cocisting of ear surgery, nose and throat surgery, cranio-maxillofacial surgery, orthopaedic surgery and spine surgery.

30. A method for manufacturing an implant, including the step of either forming said implant from a bioactive glass or coating an implant with said bioactive glass, said bioactive glass having the following composition:
SiO₂ 44-65 wt-% of the final total weight,
Na₂O 5-26 wt-% of the final total weight,
CaO 10-25 wt-% of the final total weight,
K₂O 0-15 wt-% of the final total weight,
MgO 0-6 wt-% of the final total weight,
B₂O₃ 0-4 wt-% of the final total weight, and
P₂O₅ 0-7 wt-% of the final total weight,
provided that the total amount of Na₂O and K₂O is 10-30 wt-% of the final total weight and that any source of oxygen capable of releasing oxygen in the form of molecular oxygen or reactive oxygen species, is absent.
